(19) United States
(12) Patent Application Publication
(10) Pub. No.: US 2019/0320993 A1
(43) Pub. Date: Oct. 24, 2019

(54) INFORMATION PROCESSING APPARATUS, RADIATION IMAGING APPARATUS, INFORMATION PROCESSING METHOD, AND STORAGE MEDIUM

(71) Applicant: CANON KABUSHIKI KAISHA, Tokyo (JP)

(72) Inventors: Takeshi Noda, Ebina-shi (JP); Atsushi Iwashita, Tokyo (JP)

(21) Appl. No.: 16/460,083

(22) Filed: Jul. 2, 2019

Related U.S. Application Data
(63) Continuation of application No. PCT/JP2017/039922, filed on Nov. 6, 2017.

(30) Foreign Application Priority Data
Jan. 13, 2017 (JP) 2017-004609

(51) Int. Cl. A61B 6/00; G01N 23/04

(52) U.S. Cl. CPC A61B 6/5205; G01N 2223/601; G01N 2223/401; G01N 23/04

(57) ABSTRACT

An information processing apparatus that processes information based on a radiation image capturing a subject, comprises: an average value obtainment unit configured to obtain an average value of pixel values of the radiation image; a variance value obtainment unit configured to obtain a variance value of the pixel values of the radiation image; and an arithmetic processing unit configured to calculate, based on the average value and the variance value, one of an effective atomic number and a surface density forming the subject.

### CROSS-REFERENCE TO RELATED APPLICATIONS

This application is a Continuation of International Patent Application No. PCT/JP2017/039922, filed Nov. 6, 2017, which claims the benefit of Japanese Patent Application No. 2017-004609, filed Jan. 13, 2017, both of which are hereby incorporated by reference herein in their entirety.

### BACKGROUND OF THE INVENTION

### Field of the Invention

The present invention relates to an information processing apparatus, a radiation imaging apparatus, an information processing method, and a storage medium.

### Background Art

As an imaging apparatus to be used for medical image diagnosis by radiation, a radiation imaging apparatus using a flat panel detector (to be referred to as an “FPD” hereinafter) has become popular. Since an FPD can perform digital image processing on a captured image, various kinds of applications have been developed and put into practical use.

As one such application technique, PTL 1 proposes a method of obtaining an effective atomic number by using an image captured by two types of radiation energy. An effective atomic number is defined so that a compound is regarded as a single element which has a radiation attenuation coefficient almost equal to that of the compound. Even in a case in which the component material of a subject is unknown, an approximate component material can be known by obtaining the effective atomic number.

### CITATION LIST

### Patent Literature

PTL 1: Japanese Patent Laid-Open No. 08-178873

Although PTL 1 discloses a method of calculating an effective atomic number from captured images obtained from a plurality of radiation imaging operations (imaging performed by two types of radiation energy), the measurement accuracy will degrade if motion artifacts are generated due to the movement of a subject during the plurality of radiation imaging operations, and the exposure dose of the subject can increase.

The present invention has been made in consideration of the above-problem and has as its object to provide a technique that allows an effective atomic number or a surface density of a component material of a subject to be obtained from an average value or a variance value of a radiation image obtained by one radiation imaging operation.

### SUMMARY OF THE INVENTION

An information processing apparatus according to one aspect of the present invention is an information processing apparatus that processes information based on a radiation image capturing a subject, comprising:

an average value obtainment unit configured to obtain an average value of pixel values of the radiation image;

a variance value obtainment unit configured to obtain a variance value of the pixel values of the radiation image; and

an arithmetic processing unit configured to calculate, based on the average value and the variance value, one of an effective atomic number and a surface density forming the subject.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 is a block diagram showing an example of the arrangement of a radiation imaging system according to the first embodiment;

FIG. 2 is a block diagram showing an example of the functional arrangement of an arithmetic processing unit;

FIG. 3 is a flowchart for explaining the procedure of processing in an image processing unit according to the first embodiment;

FIG. 4 is a block diagram showing the arrangement example of a radiation imaging system according to the second embodiment;

FIG. 5 is a flowchart for explaining the procedure of processing in an image processing unit according to the second embodiment;

FIG. 6 is a flowchart for explaining the procedure of processing in the image processing unit according to the second embodiment;

FIG. 7 is a view for schematically explaining tables according to the second embodiment; and

FIG. 8 is a table showing an example of an effective atomic number.

### DESCRIPTION OF THE EMBODIMENTS

Embodiments of the present invention will be described in detail below with reference to the accompanying drawings. Note that the constituent elements described in the embodiments are merely examples. The technical scope of the present invention is determined by the scope of the appended claims and is not limited by the individual embodiments to be described below.

### First Embodiment

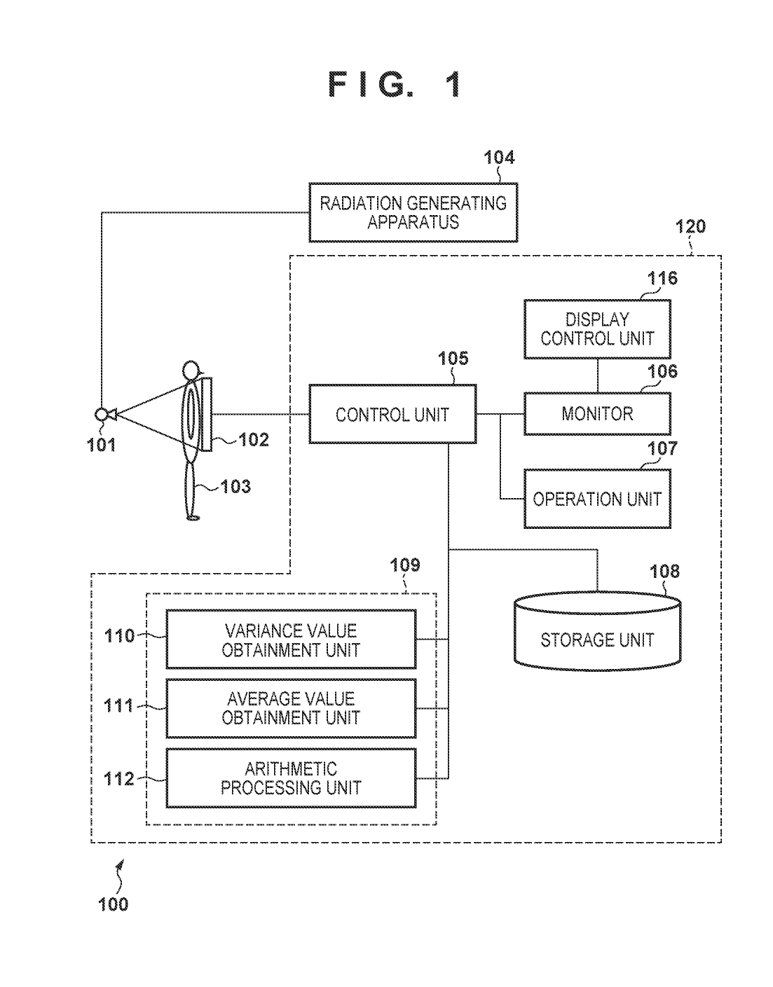

FIG. 1 is a block diagram showing an example of the arrangement of a radiation imaging system 100 according to the first embodiment of the present invention. The radiation imaging system 100 includes a radiation generating apparatus 104, a radiation tube 101, an FPD 102, and an information processing apparatus 120. Note that the arrangement of the radiation imaging system 100 may also be called simply a radiation imaging apparatus. The information processing apparatus 120 processes information based a radiation image that has captured a subject.

The radiation generating apparatus 104 generates radiation by applying a high-voltage pulse to the radiation tube 101 when an exposure switch is pressed. The radiation tube 101 irradiates a subject 103 with radiation.

When the subject 103 is irradiated with radiation from the radiation tube 101, the FPD 102 obtains a radiation image by accumulating charges based on an image signal. The FPD 102 can transfer the radiation image to the information processing apparatus 120 for each imaging operation or can store the captured image in an image storage unit in the FPD 102 without transferring the image for each image and transfer the stored images all together from the FPD 102 to the information processing apparatus 120 at a predetermined timing. The communication between the FPD 102 and the information processing apparatus 120 may be performed by wired communication or wireless communication.

The FPD 102 includes a radiation detection unit (not shown) in which a pixel array for generating a signal corresponding to the radiation is arranged. The radiation detection unit detects radiation that has been transmitted through the subject 103 as image signals. Pixels that output signals corresponding to incident light are arranged in an array (two-dimensional region) in the radiation detection unit. A photoelectric conversion element of each pixel converts light which has been converted by a fluorescent material into an image signal as an electric signal, and a capacitor of each pixel accumulates the images signal. In this manner, the radiation detection unit is configured to detect radiation transmitted through the subject 103 and obtain image signals (radiation image). A driving unit (not shown) of the FPD 102 outputs, to a control unit 105, the image signals (radiation image) read out in accordance with the instruction from the control unit 105 via an analog/digital (A/D) conversion unit.

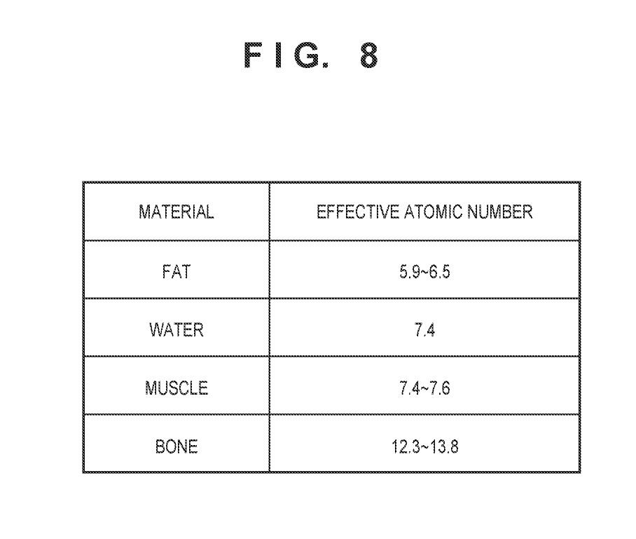

The control unit 105 includes an image processing unit 109 that processes a radiation image obtained from the FPD 102 and a storage unit 108 that stores the result of the image processing and various kinds of programs. The storage unit 108 is formed by, for example, a ROM (Read Only Memory), a RAM (Random Access Memory), or the like. The storage unit 108 can store an image output from the control unit 105, an image processed by the image processing unit 109, calculation results (for example, an effective atomic number and a surface density) obtained by the image processing unit 109, and a database (FIG. 8) in which effective atomic numbers and materials have been associated.

The image processing unit 109 includes, as functional components, a variance value obtainment unit 110, an average value obtainment unit 111, and an arithmetic processing unit 112. According to these functional components, the function of each unit is implemented by one or a plurality of CPUs (central processing units) using a program loaded from a storage unit 108. The configuration of each unit of the image processing unit 109 may be formed by an integrated circuit or the like as long as a similar function can be achieved. In addition, as the internal components of the information processing apparatus 120, it may be formed so as to include a graphic control unit such as a GPU (graphics processing unit) or the like, a communication unit such as a network card or the like, an input/output control unit that controls an input/output unit such as a keyboard, a display, or a touch panel, and the like may be included.

A monitor 106 (display unit) displays a radiation image (digital image) received by the control unit 105 from the FPD 102 and an image that has been processed by the image processing unit 109. A display control unit 116 can control the display operation of the monitor 106 (display unit). An operation unit 107 can input instructions to the image processing unit 109 and the FPD 102 and accepts the input of instructions to the FPD 102 via a user interface (not shown).

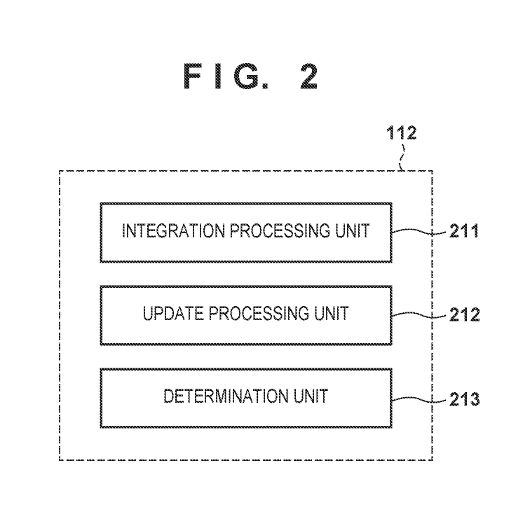

The image processing unit 109 includes, as functional components, the variance value obtainment unit 110, the average value obtainment unit 111, and the arithmetic processing unit 112, and the image processing unit 109 generates an average value image and a variance value image from a radiation image captured by the FPD 102. The arithmetic processing unit 112 calculates the effective atomic number or the surface density of a material forming the subject based on the average value and the variance value. As shown in FIG. 2, the arithmetic processing unit 112 includes, as functional components, an integration processing unit 211 for executing integration processing in the calculation processing of the effective atomic number, an update processing unit 212, and a determination unit 213.

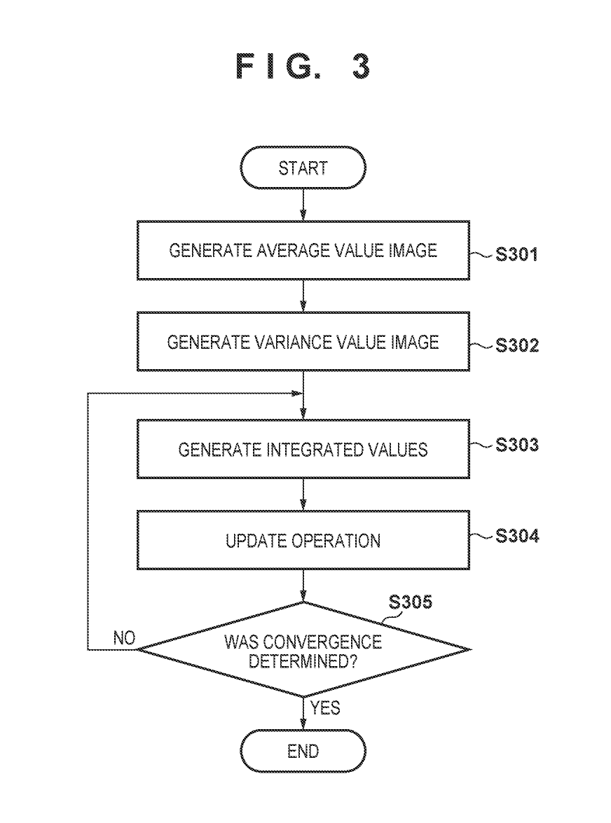

Next, the processing performed in the image processing unit 109 according to the first embodiment will be described in detail with reference to the flowchart shown in FIG. 3. The control unit 105 stores each radiation image captured by the FPD 102 in the storage unit 108 and transfers the radiation image to the image processing unit 109.

(Step S301: Generation of Average Information (Average Value Image))

In step S301, the average value obtainment unit 111 obtains an average value image which indicates an average value (average information) of pixel values obtained by dividing the pixel values of a radiation image with a subject by the pixel values of a radiation image without the subject. More specifically, the average value obtainment unit 111 obtains (generates) an average value image A(x, y) by using a radiation image M(x, y, t) with the subject and a radiation image M₀(x, y, t) without the subject (equation (1)) that have been captured by the FPD 102. Here, x and y represent coordinates of a pixel and t is an integer representing a frame number of an image captured in time series. Each bracket “<>t” represents a time average. The gain characteristic variation of the FPD 102 can be corrected by dividing the time average (average information) of the radiation image M with the subject by the time average (average information) of the radiation image M₀ without the subject. The radiation image M₀(x, y, t) without the subject is captured in advance and stored in the storage unit 108. The average value obtainment unit 111 reads out the radiation image M₀(x, y, t) without the subject from the storage unit 108 when an average value image is to be obtained, and performs arithmetic processing of equation (1).

$$A ( x , y ) = \frac{{〈 M ( x , y , t ) 〉}_{t}}{{〈 {M}_{0} ( x , y , t ) 〉}_{t}} \qquad ( 1 )$$

(Step S302: Generation of Variance Information (Variance Value Image))

In step S302, the variance value obtainment unit 110 obtains a variance value image which indicates a variance value (variance information) of pixel values obtained by dividing the pixel values of the radiation image with the subject with the pixel values of the radiation image without the subject. More specifically, the variance value obtainment unit 110 obtains (generates) a variance value image V(x, y) by using a plurality of the radiation images M(x, y, t) with the subject and the radiation image M₀(x, y, t) without the subject (equation (2)) that have been captured by the FPD 102. x and y represent coordinates of a pixel and t is an integer representing a frame number of an image captured in time series. Each bracket “<>t” represents a time average. The radiation image M₀(x, y, t) without the subject is captured in advance and stored in the storage unit 108. The variance value obtainment unit 110 reads out the radiation image M₀(x, y, t) without the subject from the storage unit 108 when a variance value image is to be obtained, and performs arithmetic processing of equation (2).

$$V ( x , y ) = \frac{{〈 {M}^{2} ( x , y , t ) 〉}_{t} - {〈 M ( x , y , t ) 〉}_{t}^{2}}{{〈 {M}_{0}^{2} ( x , y , t ) 〉}_{t} - {〈 {M}_{0} ( x , y , t ) 〉}_{t}^{2}} \qquad ( 2 )$$

(Step S303: Calculation of Parameters for Arithmetic Processing)

In step S303, the arithmetic processing unit 112 calculates parameters to be used in the arithmetic processing for obtaining the effective atomic number and the surface density of the material forming the subject. In this step, the integration processing unit 211 of the arithmetic processing unit 112 generates the following six integrated values (integration information) by using a surface density (σ_{eff}) [g/cm²] of the material forming the subject, an attenuation coefficient (μ) [cm²/g], an effective atomic number (Z_{eff}) of the material forming the subject, energy (E) of the radiation, and an energy spectrum (N(E)) of the radiation (equations (3) to (8)).

Here, a parameter Ac of equation (3) is a theoretically calculated pixel value of the radiation image and corresponds to an average value (average information). A parameter Vc of equation (4) is a theoretically calculated pixel value of the radiation image and corresponds to a variance value (variance information). That is, the parameter Ac (average information) is the first moment of the energy, and the parameter Vc is the second moment of the energy.

The parameters of equations (5) to (8) are the derivatives of the parameters Ac and Vc obtained by equations (3) and (4). The parameters obtained in step S303 are used in the arithmetic processing (update operation) which is performed in the next step. These parameters are used in an iterative calculation performed in the update operation of step S304.

$$\begin{matrix} Ac = \frac{{\int }_{0}^{∞} N ( E ) exp \{ - μ ( {Z}_{eff} , E ) {σ}_{eff} \} EdE }{{\int }_{0}^{∞} N ( E ) EdE } & ( 3 ) \\ Vc = \frac{{\int }_{0}^{∞} N ( E ) exp \{ - μ ( {Z}_{eff} , E ) {σ}_{eff} \} {E}^{2} dE }{{\int }_{0}^{∞} N ( E ) {E}^{2} dE } & ( 4 ) \\ \frac{\partial Ac}{\partial {σ}_{eff}} = \frac{{\int }_{0}^{∞} - μ ( {Z}_{eff} , E ) N ( E ) exp \{ - μ ( {Z}_{eff} , E ) {σ}_{eff} \} EdE }{{\int }_{0}^{∞} N ( E ) EdE } & ( 5 ) \\ \frac{\partial Vc}{\partial {σ}_{eff}} = \frac{{\int }_{0}^{∞} - μ ( {Z}_{eff} , E ) N ( E ) exp \{ - μ ( {Z}_{eff} , E ) {σ}_{eff} \} {E}^{2} dE }{{\int }_{0}^{∞} N ( E ) {E}^{2} dE } & ( 6 ) \\ \frac{\partial Ac}{\partial {Z}_{eff}} = \frac{{\int }_{0}^{∞} - \frac{\partial μ ( {Z}_{eff} , E )}{\partial {Z}_{eff}} {σ}_{eff} N ( E ) exp \{ - μ ( {Z}_{eff} , E ) {σ}_{eff} \} EdE }{{\int }_{0}^{∞} N ( E ) EdE } & ( 7 ) \\ \frac{\partial Vc}{\partial {Z}_{eff}} = \frac{{\int }_{0}^{∞} - \frac{\partial μ ( {Z}_{eff} , E )}{\partial {Z}_{eff}} {σ}_{eff} N ( E ) exp \{ - μ ( {Z}_{eff} , E ) {σ}_{eff} \} {E}^{2} dE }{{\int }_{0}^{∞} N ( E ) {E}^{2} dE } & ( 8 )\end{matrix}$$

Among the terms used by the integration processing unit 211 in the arithmetic processing operations of equations (3) to (8), σ_{eff} represents the surface density [g/cm²] of the material forming the subject, μ represents the attenuation coefficient [cm²/g], Z_{eff} represents the effective atomic number of the material forming the subject, E represents the energy of the radiation, and N(E) represents the energy spectrum of the radiation.

In the obtainment of the rate of change of a pixel average value or the rate of change of a pixel variance value, the integration processing unit 211 of the arithmetic processing unit 112 generates interpolation information for interpolating the attenuation coefficient by using the energy of the radiation, the atomic number of an already known element, and the attenuation coefficient corresponding to the atomic number. Also, in the obtainment of the rate of change of a pixel average value or the rate of change of a pixel variance value, the integration processing unit 211 of the arithmetic processing unit 112 obtains the rate of change per unit atomic number of the attenuation coefficient which has been interpolated based on the interpolation information.

The interpolation information and the rate of change for each unit of atomic number of the interpolated attenuation coefficient can be represented as follows in the manner of equations (9) and (10) by using the energy (E) of the radiation, the atomic number (Z) of an already known element, and the attenuation coefficient corresponding to the atomic number (Z).

The integration processing unit 211 can store the atomic number of the already known element and the corresponding attenuation coefficient in, for example, the storage unit 108, and use the atomic number of the already known element and the corresponding attenuation coefficient in the interpolation of the attenuation coefficient μ by referring to the storage unit 108. In addition, the integration processing unit 211 generates change rate information (derivative) indicating the rate of change of the attenuation coefficient μ with respect to the change of the unit effective atomic number (equation (10)). Here, in equations (9) and (10), the term ([x]) indicates a floor function which outputs the maximum integer equal to x or less with respect to a real number x.

$$\begin{matrix}μ ( Z , E ) = μ ( ( [ Z ] + 1 , E ) - μ ( [ Z ] , E ) ) ( Z - [ Z ] ) + μ ( [ Z ] , E ) & ( 9 ) \\ \frac{\partial μ ( Z , E )}{\partial Z} = μ ( [ Z ] + 1 , E ) - μ ( [ Z ] , E ) & ( 10 )\end{matrix}$$

(Step S304: Update Operation of Effective Atomic Number and Surface Density)

In step S304, the update processing unit 212 of the arithmetic processing unit 112 obtains the effective atomic number and the surface density based on equations (11). For the effective atomic number, the arithmetic processing unit 112 calculates the effective atomic number based on the rate of change of the pixel average value of the radiation image which is obtained based on the energy spectrum and the attenuation coefficient of the radiation with which the subject is irradiated with respect to the effective atomic number, the rate of change of the pixel average value with respect to the surface density, and the difference between the average value and the pixel average value.

Also, for the surface density, the update processing unit 212 of the arithmetic processing unit 112 calculates the surface density based on the rate of change of the pixel variance value of the radiation image which is obtained based on the energy spectrum of the radiation with which the subject is irradiated and the attenuation coefficient with respect to the effective atomic number, the rate of change of the pixel variance value with respect to the surface density, and the difference between the variance value and the pixel variance value.

The update processing unit 212 of the arithmetic processing unit 112 performs analysis by setting the effective atomic number, which is based on the rate of change of the pixel average value and the difference between the average value and the pixel average value, and the surface density, which is based on the rate of change of the pixel variance value and the difference between the variance value and the pixel variance value, as simultaneous equations and updates the effective atomic number and the surface density by performing an iterative operation based on the result of the analysis.

More specifically, the update processing unit 212 of the arithmetic processing unit 112 updates the effective atomic number (Z_{eff}) of the material and the surface density (σ_{eff}) of the material by performing an iterative operation based on the calculation of equations (11) below. Here, the notation of “( )” represents a matrix, and “−1” represents an inverse matrix. In addition, the exponent n represents the number of times the iterative operation is performed.

In simultaneous equations (11), the derivatives of the parameters Ac and Vc are parameters obtained by the arithmetic processing of equations (5) to (8). Also, A represents information of the average value image (average information of pixel vales of the radiation image) obtained by the arithmetic processing of equation (1), and Ac represents the average information of the pixel values of the radiation image based on the theoretical calculation of equation (3). In addition, in equations (11), V represents information of the variance value image (variance information of pixel values of the radiation image) obtained by the arithmetic processing of equation (2), and Vc represents the variance information of the pixel values of the radiation image based on the theoretical calculation of equation (4).

The update processing unit 212 obtains the effective atomic number (Z_{eff}) and the surface density (σ_{eff}) of the material forming the subject by iteratively executing the arithmetic processing of equations (11) by performing an iterative calculation based on, for example, the Newton-Raphson method. At this time, an arbitrary value such as a zero value or the like may be set as the initial value of the operation.

$$( \begin{matrix}{Z}_{eff}^{n + 1} \\ {σ}_{eff}^{n + 1}\end{matrix} ) = ( \begin{matrix}{Z}_{eff}^{n} \\ {σ}_{eff}^{n}\end{matrix} ) + {( \begin{matrix}\frac{\partial Ac}{\partial {Z}_{eff}^{n}} & \frac{\partial Ac}{\partial {σ}_{eff}^{n}} \\ \frac{\partial Vc}{\partial {Z}_{eff}^{n}} & \frac{\partial Vc}{\partial {σ}_{eff}^{n}}\end{matrix} )}^{- 1} ( \begin{matrix}A - {Ac}^{n} \\ V - {Vc}^{n}\end{matrix} ) \qquad ( 11 )$$

(Step S305: Convergence Determination)

In step S305, the determination unit 213 of the arithmetic processing unit 112 determines the convergence of the effective atomic number and the surface density that have been updated by the update processing unit 212. The determination unit 213 determines whether the effective atomic number (Z_{eff}) of the material and the surface density (σ_{eff}) of the material, which have been updated by the iteration calculation performed in step S304, have converged. Various kinds of methods can be used as the convergence method to make this determination. For example, in a case in which the difference between an nth update operation result and an (n+1)th update operation result is equal to a predetermined threshold or less upon comparing these two update operation results, the determination unit 213 can determine that the (n+1)th update operation result has converged because predetermined calculation accuracy has been obtained. Alternatively, the iteration count of the update operation by the update processing unit 212 can be obtained, and the determination unit 213 can determine that the update operation result has converged when the update operation has been executed for a predetermined iteration count.

If the determination unit 213 determines that the update operation result has not converged in the convergence determination performed in step S305 (NO in step S305), the process returns to step S303, and the generation processing of integrated values (calculation of the parameters to be used in the arithmetic processing) is executed again. On the other hand, if the determination unit 213 determines that the update operation result has converged in the convergence determination performed in step S305 (YES in step S305), the arithmetic processing unit 112 outputs the converged effective atomic number or the converged surface density as the effective atomic number or the surface density of the material forming the subject, and the processing of the image processing unit 109 ends.

According to this embodiment, the effective atomic number and the surface density of the material forming the subject can be obtained from the average value (average information) and the variance information (variance information) of the radiation image.

The image processing unit 109 can generate an image (combined image) associating the result of the image processing (the calculation result of at least one of the effective atomic number and the surface density) with the image. The display control unit 116 can associate (combine), for example, the effective atomic number or the surface density with the radiation image and cause the monitor 106 (display unit) to display the resultant image as the image processing result.

By performing display control in this manner, it becomes possible to visualize and display the correspondence relationship between the radiation image and the effective atomic number (the atomic number of the element having an attenuation coefficient equal to the material) of the material forming the subject in the radiation image. For example, the storage unit 108 can store a database in which materials are associated with their respective effective atomic numbers such as that shown in FIG. 8, and elements forming the parts of the subject of the radiation image can be displayed on the monitor 106 based on the calculated effective atomic numbers. As a result, it becomes possible to confirm whether an instrument has been left behind after a surgery and to improve the visibility of a lesion or a contrast agent, and thus facilitate support for a doctor's diagnosis or an imaging operation performed by a radiographer.

### Second Embodiment

In this embodiment, an arrangement in which an effective atomic number is obtained by referring to a table (a two-dimensional effective atomic number table) indicating the relationship between on a variance value and an average value of pixel values of a radiation image and the effective atomic number of a material forming a subject will be described. In addition, an arrangement in which a surface density is obtained by referring to a table (a two-dimensional surface density table) indicating the relationship between the variance value and the average value of pixel values of the radiation image and the surface density of the material forming the subject will be described.

In the following description, a description of parts similar to the first embodiment will be omitted, and only component parts specific to the second embodiment will be described. Compared to an arrangement in which the effective atomic number and the surface density are obtained analytically by obtaining an average value image and a variance value image from the radiation image, the arrangement according to this embodiment is capable of performing high-speed operations when it is implemented. Thus, the arrangement according to this embodiment is effective when the effective atomic number and the surface density are to be calculated in a moving image capturing operation.

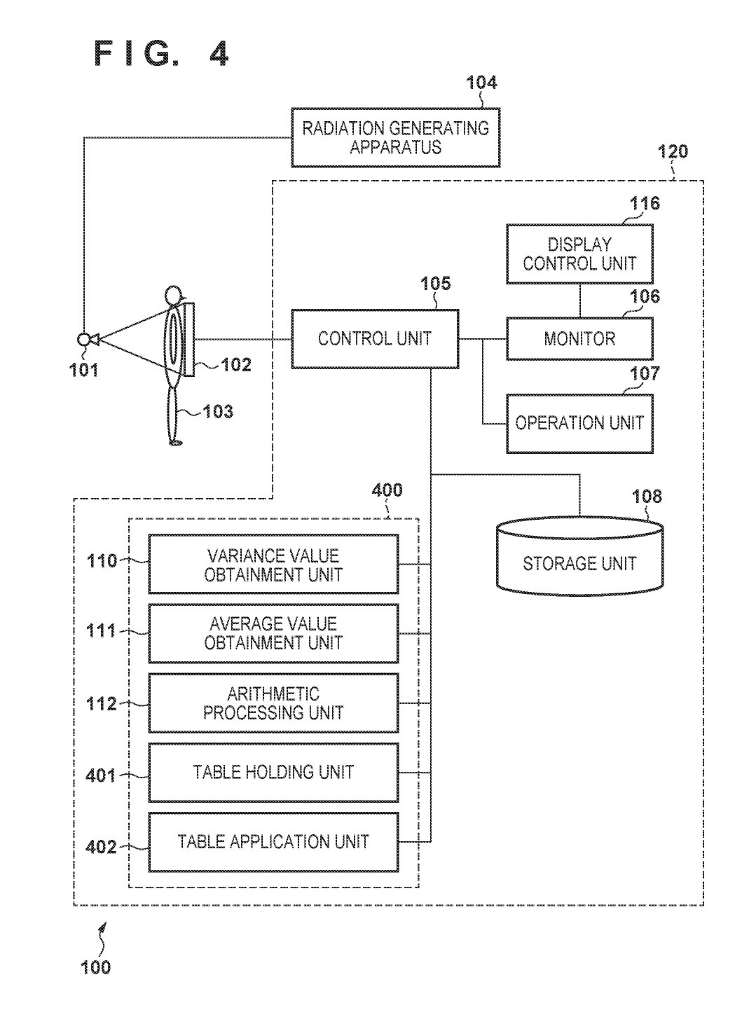

FIG. 4 is a block diagram showing an example of the arrangement of a radiation imaging system 100 according to the second embodiment of the present invention. This embodiment is different from the first embodiment in that an image processing unit 400 includes a table holding unit 401 and a table application unit 402.

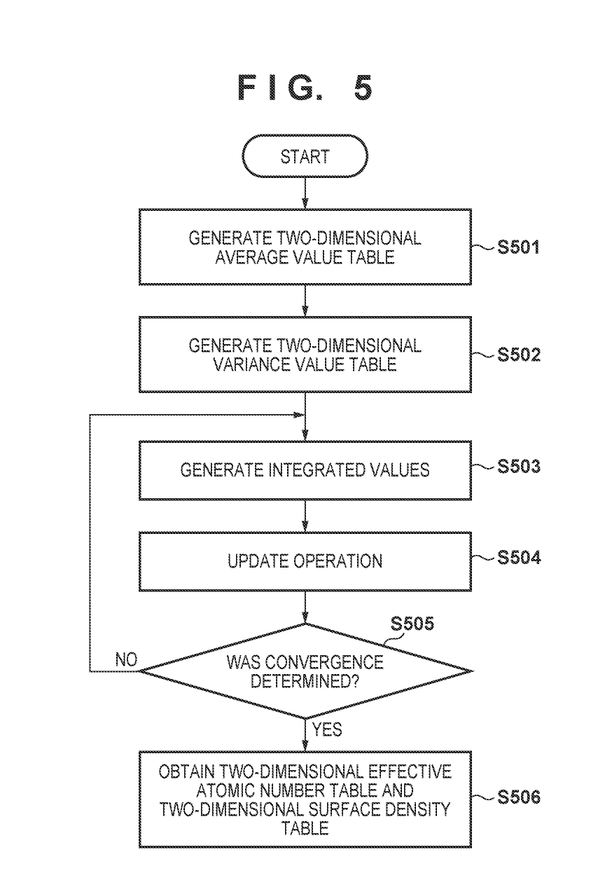

Next, the processing of image processing unit 400 according to the second embodiment will be described in detail next with reference to the flowchart shown in FIG. 5. A control unit 105 stores a radiation image captured by an FPD 102 in a storage unit 108 and transfers the radiation image to the image processing unit 400.

(Step S501: Generation of Two-Dimensional Average Value Table)

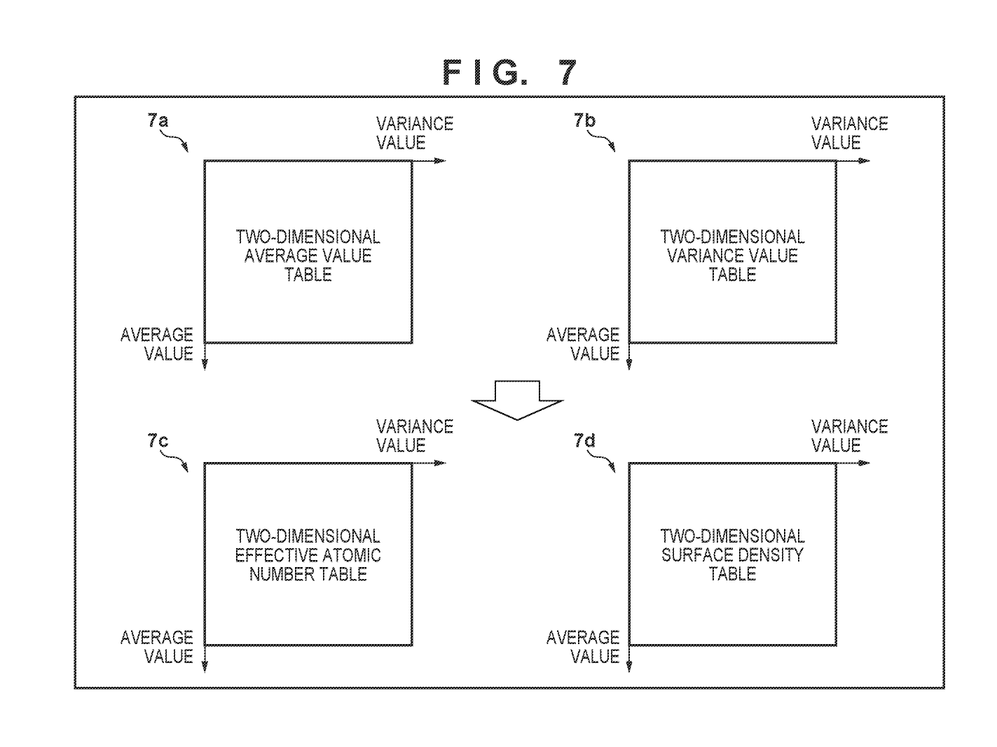

In step S501, before the actual imaging is to be started in this embodiment, an integration processing unit 211 of an arithmetic processing unit 112 generates a two-dimensional average value table. A two-dimensional average value table is generated as, for example, a two-dimensional matrix table in which the X-axis (abscissa) indicates the variance value and the Y-axis (ordinate) indicates the average value in the manner of a table 7a shown in FIG. 7. The integration processing unit 211 generates the two-dimensional average value table based on a range of values of 0.0 to 0.1 that an average value Ac (theoretical value) of equation (3) may take and on a range of values of 0.0 to 1.0 that a variance value Vc (theoretical value) of equation (4) may take. That is, a table is generated so that, as each matrix element of the two-dimensional average value table shown by the table 7a of FIG. 7, the average value Ac is embedded from values of 0.0 to 1.0 in the Y-axis (ordinate) direction and a predetermined value of the variance value Vc is set in correspondence with the average value Ac in the X-axis (abscissa) direction. For example, values ranging from 0.0 to 1.0 that the average value Ac (theoretical value) can take are arranged as the matrix elements in correspondence with the variance value Vc having a value of 0.0. In a similar manner, values ranging from 0.0 to 1.0 that the average value Ac (theoretical value) can take are arranged as the matrix elements in correspondence with the variance value Vc having a value of 1.0.

Note that the integration processing unit 211 can generate the two-dimensional average value table as a one-dimensional average value table that associates the variance value Vc and the average value Ac. In this case, the integration processing unit 211 need only appropriately associate the one-dimensional average value table in accordance with the matrix elements of a two-dimensional effective atomic number table and the matrix elements of a two-dimensional surface density table (both tables to be described later).

(Step S502: Generation of Two-Dimensional Variance Value Table)

In step S502, the integration processing unit 211 generates a two-dimensional variance value table. A two-dimensional variance value table is generated as, for example, a two-dimensional matrix table in which the X-axis (abscissa) indicates the variance value and the Y-axis (ordinate) indicates the average value in the manner of a table 7b shown in FIG. 7. The integration processing unit 211 generates the two-dimensional variance value table based on the range of values of 0.0 to 0.1 that the average value Ac (theoretical value) of equation (3) can take and on the range of values of 0.0 to 1.0 that the variance value Vc (theoretical value) of equation (4) can take. That is, a table is generated so that, as each matrix element of the two-dimensional variance value table shown by the table 7b of FIG. 7, the variance values Vc are embedded from 0.0 to 1.0 in the X-axis (abscissa) direction and a predetermined value of the average value Ac is set in correspondence with the average value Vc in the Y-axis (ordinate) direction. For example, values ranging from 0.0 to 1.0 that the average value Vc (theoretical value) may take are arranged as the matrix elements in correspondence with the average value Ac having a value of 0.0. In a similar manner, values ranging from 0.0 to 1.0 that the variance value Vc (theoretical value) may take are arranged as the matrix elements in correspondence with the average value Ac having a value of 1.0.

Note that the integration processing unit 211 can generate the two-dimensional variance value table as a one-dimensional variance value table that associates the variance value Vc and the average value Ac. In this case, the integration processing unit 211 need only appropriately associate the one-dimensional variance value table in accordance with the matrix elements of the two-dimensional effective atomic number table and the matrix elements of the two-dimensional surface density table (both tables to be described later).

(Steps S503 to S505: Generation of Integrated Values, Update Operation, and Convergence Determination)

The process of step S503 corresponds to calculation processing (generation of integrated values) of parameters to be used for the arithmetic processing of step S303. The process of step S503 corresponds to update operation processing of the effective atomic number and the surface density of step S304. In addition, the process of step S505 corresponds to the convergence determination processing of step S305.

Here, in the arithmetic processing of equations (11), the information of the two-dimensional average value table (the table 7a of FIG. 7) generated in step S501 is used as the information of an average value image A which indicates the average information of the pixel values of the radiation image. Also, the information of the two-dimensional variance value table (the table 7b of FIG. 7) generated in step S502 is used as the information of a variance value image V which indicates the variance information of the pixel values of the radiation image.

(Step S506: Obtainment of Two-Dimensional Tables)

In step S506, the update processing unit 212 obtains an effective atomic number (Z_{eff}) and a surface density (σ_{eff}) of the material forming the subject iteratively executing the arithmetic processing of equation 11 by performing an iterative calculation. As a result of the iterative operation, the update processing unit 212 obtains a two-dimensional effective atomic number table (a table 7c of FIG. 7) associating the effective atomic number (Z_{eff}) with the variance value (σ_{eff}) and the average value. The update processing unit 212 also obtains, as a result of the iterative operation, a two-dimensional surface density table (a table 7d of FIG. 7) associating the surface density with the variance value and the average value.

The two-dimensional effective atomic number table (the table 7c of FIG. 7) is generated as a two-dimensional matrix table in which the X-axis (abscissa) indicates the variance value and the Y-axis (ordinate) indicates the average value, and it is possible to obtain an effective atomic number corresponding to a variance value and an average value if the variance value and the average value are obtained. In a similar manner, the two-dimensional surface density table (the table 7d of FIG. 7) is generated as a two-dimensional matrix table in which the X-axis (abscissa) indicates the variance value and the Y-axis (ordinate) indicates the average value, and it is possible to obtain a surface density corresponding to a variance value and an average value if the variance value and the average value are obtained. The table holding unit 401 holds the two-dimensional effective atomic number table (the table 7c of FIG. 7) and the two-dimensional surface density table (the table 7d of FIG. 7) that have been generated.

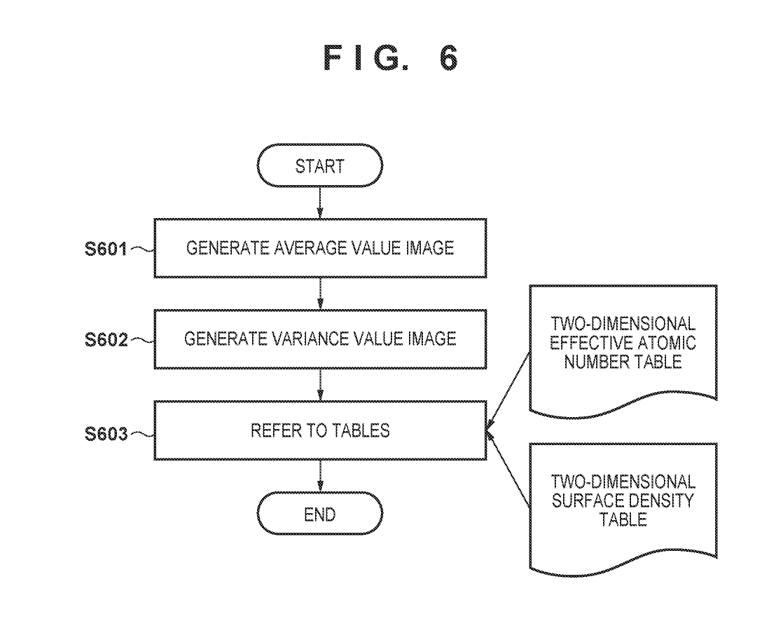

The processes up to this point are a preparation to start the actual imaging operation. The procedure of processing of the actual imaging operation will be described next with reference to the flowchart shown in FIG. 6.

(Step S601: Generation of Average Information (Average Value Image))

In step S601, an average value obtainment unit 111 obtains an average value image which indicates the average value (average information) of the pixel values of the radiation image. This processing is similar to the processing of step S301 of FIG. 3, and the average value obtainment unit 111 obtains (generates) an average value image A(x, y) by using a radiation image M(x, y, t) with the subject and a radiation image M₀(x, y, t) without the subject that have been captured by the FPD 102 (equation (1)).

(Step S602: Generation of Variance Information (Variance Value Image))

In step S602, a variance value obtainment unit 110 obtains a variance value image which indicates the variance value (variance information) of the pixel values of the radiation image. This processing is similar to the processing of step S302 of FIG. 3, and the variance value obtainment unit 110 obtains (generates) a variance value image V(x, y) by using a plurality of the radiation images M(x, y, t) with the subject and the radiation image M₀(x, y, t) without the subject that have been captured by the FPD 102 (equation (2)).

(Step S603: Referring of Two-Dimensional Tables)

In step S603, the arithmetic processing unit 112 refers to the two-dimensional effective atomic number table (the table 7c of FIG. 7) and the two-dimensional surface density table (the table 7d of FIG. 7) which are stored in the table holding unit 401, and generates an effective atomic number image indicating the distribution of the effective atomic number and a surface density image indicating the distribution of the surface density that correspond to the pixel value (average information) of the average value image A(x, y) and the pixel value (variance information) of the variance value image V(x, y), respectively. Each pixel value of the effective atomic number image indicates the effective atomic number, and each pixel value of the surface density image indicates the surface density. Note that when arithmetic processing unit 112 refers to the two-dimensional effective atomic number table (the table 7c of FIG. 7) and the two-dimensional surface density table (the table 7d of FIG. 7), the corresponding average value and the variance value may not always be present on the two-dimensional tables, respectively. In such a case, the arithmetic processing unit 112 can obtain and output the effective atomic number and the surface density by performing an interpolation operation using an already known average value and an already known variance value stored in the two-dimensional tables, respectively. For example, the arithmetic processing unit 112 may refer to each table by using bilinear interpolation (equations (12) and (13)) as shown below. Note that the interpolation operation method is not limited to bilinear interpolation, and it is possible to use, for example, nearest neighbor interpolation, spline interpolation, bicubic interpolation, or the like.

$$\begin{matrix}{Z}_{out} = {}_{WAPWv} Z ( [ A ] + 1 , [ V ] ) + {}_{WAWv} Z ( [ A ] , [ V ] ) + {}_{WAWvP} Z ( [ A ] , [ V ] + 1 ) + {}_{WAPWvP} Z ( [ A ] + 1 , [ V ] + 1 ) & ( 12 ) \\ {D}_{out} = {}_{WAPWv} D ( [ A ] + 1 , [ V ] ) + {}_{WAWv} D ( [ A ] , [ V ] ) + {}_{WAWvP} D ( [ A ] , [ V ] + 1 ) + {}_{WAPWvP} D ( [ A ] + 1 , [ V ] + 1 ) & ( 13 )\end{matrix}$$

where weight coefficients of the interpolation processing in equations (12) and (13) are

w_{AP}=A−[A]

w_{A}=1−w_{AP}

w_{VP}=V−[V]

w_{V}=1−w_{VP}

Z_{OUT} of equation (12) represents a pixel value (effective atomic number) of the effective atomic number image indicating the distribution of the effective atomic number obtained by the interpolation processing, and D_{OUT} represents a pixel value (surface density) of the surface density image indicating the distribution of the surface density obtained by the interpolation processing. A represents a value of the corresponding two-dimensional average value table, and V represents a value of the corresponding two-dimensional variance value table value, and each notation of ([x]) in equations (12) and (13) represents a floor function that outputs a maximum integer equal to or less than x with respect to a real number x.

According to this embodiment, the effective atomic number and the surface density of the material forming the subject can be obtained from the average information and the variance information of the radiation image. According to the processing of this embodiment, the effective atomic number image indicating the distribution of the effective atomic number and the surface density image indicating the distribution of the surface density can be generated by referring to two-dimensional tables (the table 7a of FIG. 7 to the table 7d of FIG. 7) without requiring integration operations such as equations (3) to (8) of the first embodiment and an iterative operation such as equations (11) to be executed for each pixel of a captured image. Since the operation load required for integration operations such as equations (3) to (8) and an iterative operation such as equations (11) can be reduced, the effective atomic number image indicating the distribution of the effective atomic number and the surface density image indicating the distribution of the surface density can be generated at a higher speed than the processing according to the first embodiment. In particular, this embodiment is very effective when the processing is to be performed in real time such as when fluoroscopy (moving image capturing operation) using radiation is to be performed.

Although an arrangement in which the average value and the variance value were used as statistical information in the first embodiment and the second embodiment, the present invention is not limited to this example, and it is possible to use statistical information related to the third moment or the fourth moment. For example, it is possible to use statistical information (skewness) obtained by normalizing the third moment about the average value by standard deviation or statistical information (kurtosis) obtained by normalizing the fourth moment about the average value by standard deviation. The information processing apparatus that processes information based on a radiation image capturing the subject includes an obtainment unit, which obtains a plurality of pieces of statistical information of different pixel values of the radiation image, and an arithmetic processing unit, which calculates the effective atomic number or the surface density of the material forming the subject based on the plurality of pieces of statistical information.

Note that the present invention is not limited to the above-described embodiments, and various changes and modifications can be made without departing from the scope of the present invention. The present invention can adopt an embodiment in the form of, for example, a system, apparatus, method, program, or storage medium. More specifically, the present invention may be applied to a system constituted by a plurality of devices, or an apparatus comprising a single device.

According to the present invention, the effective atomic number and the surface density of a material forming a subject can be obtained from an average value and a variance value of a radiation image.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information processing apparatus that processes information based on a radiation image capturing a subject, comprising:
an average value obtainment unit configured to obtain an average value of pixel values of the radiation image;
a variance value obtainment unit configured to obtain a variance value of the pixel values of the radiation image; and
an arithmetic processing unit configured to calculate, based on the average value and the variance value, one of an effective atomic number and a surface density forming the subject.

2. The information processing apparatus according to claim 1, wherein the arithmetic processing unit calculates the effective atomic number based on a rate of change of a pixel average value of a radiation image which is obtained based on an energy spectrum and an attenuation coefficient of radiation with which the subject is irradiated, a rate of change of the pixel average value with respect to the surface density, and a difference between the average value and the pixel average value.

3. The information processing apparatus according to claim 2, wherein the arithmetic processing unit calculates the surface density based on a rate of change of a pixel variance value of a radiation image which is obtained based on the energy spectrum of the radiation with which the subject is irradiated and the attenuation coefficient, a rate of change of the pixel variance value with respect to the surface density, and a difference between the variance value and the pixel variance value.

4. The information processing apparatus according to claim 3, wherein in the obtainment of one of the rate of change of the pixel average value and the rate of change of the pixel variance value, the arithmetic processing unit generates interpolation information for interpolating the attenuation coefficient by using the energy of the radiation, an atomic number of an already known element, and an attenuation coefficient corresponding to the atomic number.

5. The information processing apparatus according to claim 4, wherein in the obtainment of one of the rate of change of the pixel average value and the rate of change of the pixel variance value, the arithmetic processing unit obtains a rate of change per unit atomic number of the attenuation coefficient interpolated based on the interpolation information.

6. The information processing apparatus according to claim 4, wherein the arithmetic processing unit analyzes, as simultaneous equations,
the effective atomic number obtained based on the rate of change of the pixel average value and the difference between the average value and the pixel average value, and
the surface density obtained based on the rate of change of the pixel variance value and the difference between the variance value and the pixel variance value, and updates the effective atomic number and the surface density by performing an iterative operation based on a result of the analysis.

7. The information processing apparatus according to claim 6, further comprising:
a determination unit configured to determine convergence of effective atomic number and the surface density that have been updated,
wherein the arithmetic processing unit outputs, one of the effective atomic number and the surface density that have converged as one of the effective atomic number and the surface density of the material forming the subject.

8. An information processing apparatus that processes information based on a radiation image capturing a subject, comprising:
a storage unit configured to store a table showing a relationship between an effective atomic number of a material forming the subject and a variance value and an average value of pixel values of the radiation image;
an average value obtainment unit configured to obtain the average value of the pixel values of the radiation image;
a variance value obtainment unit configured to obtain the variance value of the pixel values of the radiation image; and
an arithmetic processing unit configured to obtain the effective atomic number from the table based on the average value and the variance value.

9. An information processing apparatus that processes information based on a radiation image capturing a subject, comprising:
a storage unit configured to store a table showing a relationship between a surface density of a material forming the subject and a variance value and an average value of pixel values of the radiation image;
an average value obtainment unit configured to obtain the average value of the pixel values of the radiation image;
a variance value obtainment unit configured to obtain the variance value of the pixel values of the radiation image; and
an arithmetic processing unit configured to obtain the surface density from the table based on the average value and the variance value.

10. The information processing apparatus according to claim 1, wherein the average value obtainment unit obtains an average value image which indicates average information of pixel values obtained by dividing pixel values of a radiation image with the subject by pixel values of a radiation image without the subject.

11. The information processing apparatus according to claim 1, wherein the variance value obtainment unit obtains a variance value image which indicates variance information of pixel values obtained by dividing pixel values of a radiation image with the subject by pixel values of a radiation image without the subject.

12. The information processing apparatus according to claim 1, further comprising:
a display control unit configured to cause a display unit to display one of the effective atomic number and the surface density in association with the radiation image.

13. An information processing apparatus that processes information based on a radiation image capturing a subject, comprising:
an unit configured to obtain a plurality of pieces of statistical information of different pixel values of the radiation image; and
an arithmetic processing unit configured to calculate, based on the plurality of pieces of statistical information, one of an effective atomic number and a surface density of a material forming the subject.

14. A radiation imaging apparatus that includes
an imaging unit configured to capture a radiation image, and
an information processing apparatus that processes information based on a radiation image capturing a subject by the imaging unit, wherein
the information processing apparatus includes
an average value obtainment unit configured to obtain an average value of pixel values of the radiation image,
a variance value obtainment unit configured to obtain a variance value of the pixel values of the radiation image, and
an arithmetic processing unit configured to calculate, based on the average value and the variance value, one of an effective atomic number and a surface density of a material forming the subject.

15. An information processing method of an information processing apparatus that processes information based on a radiation image capturing a subject, the method comprising:
a step of causing an average value obtainment unit to obtain an average value of pixel values of the radiation image;
a step of causing a variance value obtainment unit to obtain a variance value of the pixel values of the radiation image; and
a step of causing an arithmetic processing unit to calculate, based on the average value and the variance value, one of an effective atomic number and a surface density of a material forming the subject.

16. A computer-readable storage medium storing a program for causing a computer to function as each unit of an information processing apparatus that processes information based on a radiation image capturing a subject, the information processing apparatus comprising:
an average value obtainment unit configured to obtain an average value of pixel values of the radiation image;
a variance value obtainment unit configured to obtain a variance value of the pixel values of the radiation image; and
an arithmetic processing unit configured to calculate, based on the average value and the variance value, one of an effective atomic number and a surface density forming the subject.
